(19) United States
(12) Patent Application Publication
(10) Pub. No.: US 2025/0270335 A1
(43) Pub. Date: Aug. 28, 2025

(54) METHODS OF TREATING INFLAMMATION

(71) Applicant: Monash University, Clayton (AU)

(72) Inventors: Remy Robert, Clayton (AU); Charles Reay MACKAY, Clayton (AU)

(21) Appl. No.: 19/036,353

(22) Filed: Jan. 24, 2025

Related U.S. Application Data
(63) Continuation of application No. 17/280,537, filed on Mar. 26, 2021, now abandoned.

(30) Foreign Application Priority Data
Oct. 9, 2018 (AU) 2018903814

(51) Int. Cl. C07K 16/28; A61K 39/00; A61P 1/16

(52) U.S. Cl. CPC C07K 16/2866; A61P 1/16; A61K 2039/505; C07K 2317/54; C07K 2317/55; C07K 2317/56; C07K 2317/626; C07K 2317/732

(57) ABSTRACT

The invention relates to the use of anti-CXCR3 for detection and therapy of various conditions. The invention also relates to compositions comprising antibodies for binding to CXCR3 and their use.

### CROSS-REFERENCE TO RELATED APPLICATIONS

This application is a continuation of U.S. patent application Ser. No. 17/280,537, filed Mar. 26, 2021, which is a national stage application, filed under 35 U.S.C. 371, of International Application No. PCT/AU2019/050561, filed on May 31, 2019, which claims priority from, and the benefit of Australian Provisional Application No. 2018903814, filed on Oct. 9, 2018. The contents of each of the aforementioned patent applications are incorporated herein by reference in their entireties.

### FIELD OF THE INVENTION

The invention relates to methods and compositions for treating or preventing conditions associated with CXCR3 expression, in particular non-alcoholic steatohepatitis (NASH).

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “FRPA-021_C01 US_SeqList.xml”. The XML file is 14,615 bytes, created on Jan. 24, 2025, and is being submitted electronically via USPTO Patent Center.

### BACKGROUND OF THE INVENTION

Chemokines or chemoattractant cytokines comprise a family of inducible secreted molecules of small molecular weight (˜8-10 KDa) which typically function as activators and chemoattractants to leukocytes and can modulate angiogenesis, wound healing, and tumorigenesis Most of the knowledge of chemokine functions is derived from research on the immune system due to their implication in regulation of immune coordination and inflammation. Roles involving many other biological systems are slowly being elucidated.

Chemokines are typically classified into four subfamilies according to the number of conserved cysteine residues in their amino terminus. Most chemokines fit into two main subfamilies with four cysteine residues. These subfamilies are typically classified according to the presence or absence of an amino acid between the two amino terminus cysteine residues, and are thus named CC and CXC chemokines. CXC chemokines, which are typically restricted to higher vertebrates, are usually further classified according to the presence or absence of a glutamate-lysine-arginine (ELR) motif on their amino terminus adjacent to the first cysteine residue.

Chemokine receptors, such as CXCR3, are G-protein coupled receptors (GPCRs) typically linked to pertussis toxin (PTX) sensitive Gi proteins. The CXCR's N-terminus domain is thought to be important for determining ligand binding specificity.

CXCR3 is also known as G protein-coupled receptor 9 (GPR9), CD182, CD183, CKR-L2, CMKAR3, GPR9, IP10-R, Mig-R, MigR, and C-X-C motif chemokine receptor 3, and is a 38 kDa seven transmembrane protein. CXCR3 is expressed primarily on activated T lymphocytes and NK cells, and some epithelial cells. CXCR3 is preferentially expressed on Th1 cells (whereas Th2 cells favor the expression of CCR3 and CCR4). CXCR3 ligands that attract Th1 cells can concomitantly block the migration of Th2 cells in response to CCR3 ligands, thus enhancing the polarization of effector T cell recruitment. CXCR3 is able to regulate leukocyte trafficking. Binding of chemokines to CXCR3 induces various cellular responses, most notably integrin activation, cytoskeletal changes and chemotactic migration. CXCR3-ligand interaction attracts Th1 cells and promotes Th1 cell maturation.

CXCR3 is expressed in in vitro cultured effector/memory T cells, and in T cells present in many types of inflamed tissues. In addition, CXCL9, CXCL10 and CXCL11 are commonly produced by local cells in inflammatory lesion, suggesting that CXCR3 and its chemokines participate in the recruitment of inflammatory cells. CXCR3 has been implicated in cancer, wound healing, responses to infectious disease and tissue transplantation and autoimmune diseases including: atherosclerosis, multiple sclerosis, pulmonary fibrosis, type 1 diabetes, autoimmune myasthenia gravis, nephrotoxic nephritis, acute cardiac allograft rejection and Celiac Disease.

There exists a need for new and/or improved methods to treat diseases associated with CXCR3 activation.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect the present invention provides a method for treating a condition associated with effector and/or memory T cells in a subject, the method comprising, consisting essentially of or consisting of administering to the subject a depleting antigen binding site that binds to CXCR3, thereby treating a condition associated with effector and/or memory T cells in the subject.

In another aspect the present invention also provides a method for treating fatty liver disease in a subject, the method comprising, consisting essentially of or consisting of administering to the subject a depleting antigen binding site that binds to CXCR3, thereby treating the fatty liver disease in the subject.

In a further aspect, the present invention provides a method for preventing or delaying the onset of fatty liver disease in a subject, the method comprising, consisting essentially of or consisting of administering to the subject a depleting antigen binding site that binds to CXCR3, thereby preventing or delaying the onset the fatty liver disease in the subject.

The fatty liver disease may be associated, caused by or the result of alcoholism. Alternatively, the fatty liver disease may be associated, caused by or the result of a non-alcohol dietary cause (non-alcohol fatty liver disease, NAFLD). The NAFLD may be simple steatosis or may be simple steatosis including one or more signs of inflammation and fibrosis.

The fatty liver disease may be characterised by liver steatosis, elevated circulating or liver triglycerides and/or elevated circulating or liver cholesterol levels.

In a further aspect, the present invention provides a method for treating non-alcoholic steatohepatitis (NASH) in a subject, the method comprising, consisting essentially of or consisting of a depleting antigen binding site that binds to CXCR3, thereby treating NASH in the subject.

In another aspect, the present invention also provides a method for preventing or delaying the onset of non-alcoholic steatohepatitis (NASH) in a subject, the method comprising, consisting essentially of or consisting of a depleting antigen binding site that binds to CXCR3, thereby preventing or delaying the onset of NASH.

In another aspect, the invention also provides a method of reducing or treating inflammation in a subject at risk of, or having an autoimmune disease, the method comprising, consisting essentially of or consisting of a depleting antigen binding site that binds to CXCR3, thereby reducing or treating inflammation in the subject.

Reducing or treating inflammation may include reducing the proportion of one or more pro-inflammatory cytokines in the individual. Further, reducing or treating inflammation may include increasing the proportion of one or more anti-inflammatory cytokines in the subject.

In one aspect the invention provides a method for inhibiting the recruitment or migration of CXCR3+ cells to an inflammation site in a subject, comprising administering to the subject an effective amount of a depleting antigen binding site that binds to CXCR3, thereby inhibiting the recruitment or migration of CXCR3+ cells to an inflammation site in the subject.

In any aspect of the present invention, the antigen binding site that binds to CXCR3 is a depleting antibody. The depleting antibody has the capacity to cause or mediate a reduction in activity or viability of a cell expressing CXCR3. Typically the depleting antibody has the capacity to induce antibody-dependent cell-mediated cytotoxicity (ADCC).

The antigen binding site may have been modified to provide the ability to deplete a cell, or may have been modified to increase the existing capacity of the antigen binding site to deplete a cell.

In some embodiments, the cells are CXCR3+/CD4+ T cells, CXCR3+/CD8+ T cells and/or CXCR3+/CD19+ B cells. In further embodiments, the subject has received, is receiving or is to receive a graft or transplant.

In another aspect, the present invention provides a method of treating or preventing the accumulating of lipid deposits in the liver of a subject, the method comprising administering a depleting antigen binding site that binds to CXCR3 to the subject, thereby treating or preventing the accumulating of lipid deposits in the liver of a subject.

In another aspect, the invention provides a method of treating or preventing fatty liver disease in a subject, the method comprising administering a depleting antigen binding site that binds to CXCR3 to the subject, thereby treating or preventing fatty liver disease in the subject. The fatty liver disease may be non-alcoholic fatty liver disease (NAFLD) or caused by or is the result alcoholism.

In another aspect, the invention provides a method of treating or preventing steatohepatitis in a subject, the method comprising administering a depleting antigen binding site that binds to CXCR3 to the subject, thereby treating or preventing steatohepatitis. The steatohepatitis may be alcoholic steatohepatitis (ASH) or non-alcoholic steatohepatitis (NASH). Preferably, the steatohepatitis is NASH.

In another aspect, the invention provides a method for inhibiting graft or transplant rejection in a subject that has received, is receiving or is to receive a graft or transplant comprising administering to the subject an effective amount of a depleting antigen binding site that binds to CXCR3, thereby inhibiting graft or transplant rejection in the subject.

In some embodiments of the methods of the invention, the graft is an allograft or xenograft, or the transplant is an allotransplant or xenotransplant. In particular examples, the graft or transplant is selected from among a heart, kidney, lung, liver, pancreas, pancreatic islets, brain tissue, stomach, large intestine, small intestine, cornea, skin, trachea, bone, bone marrow, muscle and bladder graft or transplant. In some instances, the antibody or antigen-binding fragment is administered to the subject before, at the same time and/or after the subject has received the graft or transplant.

In one embodiment of the methods of the invention, the antibody or antigen-binding fragment is administered to the subject two or more times. In some examples, the methods further comprise administering to the subject one or more other therapeutic agents, such as a cytokine (e.g. interleukin 2), chemokine or antibody. In particular embodiments, the therapeutic agent is an immunosuppressive agent, for example, a glucocorticoid, cyclosporine, rapamycin, voclosporin, sirolimus, everolimus. tacrolimus, mycophenolate, mofetil, mycophenolic acid, mizoribine, an S1P-R agonist, a malononitrilamide, an anti-CD3 antibody, an anti-CD25 antibody, an anti-CD52 antibody, an anti-CD20 antibody or an anti-tumor necrosis factor antibody.

Also provided are uses of a depleting antigen binding site that binds to CXCR3 for the preparation of a medicament for depleting CXC3+ cells; inhibiting the recruitment or migration of CXCR3+ cells to an inflammation site; and/or the preparation of a medicament for inhibiting graft rejection in a subject that has received, is receiving or is to receive a graft.

In another aspect, the present invention provides a method of treating fibrosis in a subject, the method comprising administering an antigen binding site as described herein to the subject, thereby treating fibrosis.

In any aspect of the present invention the antigen binding site that binds to or specifically binds to CXCR3 and inhibits CXCR3 activity.

Preferably, the antigen binding site comprises an antigen binding domain of an antibody, the antigen binding domain binds to or specifically binds to CXCR3 and inhibits CXCR3 activity.

The CXCR3 activity that may be inhibited by any antigen binding site of the invention includes: ligand binding to CXCR3; ligand induced conformational change of CXCR3; CXCR3 activation; G protein activation; CXCR3 mediated cell signalling; a CXCR3 mediated cell migratory, inflammatory, tumour growth, angiogenic or metastatic response in vitro or in vivo; CXCR3 mediated tumour cell growth; and/or CXCR3 mediated recruitment of inflammatory cells, leukocyte (e.g. neutrophil, eosinophil, mast cell or T cell) migration, integrin activation, chemotactic migration and Th1 cell maturation.

Preferably, the antigen binding site as described herein binds to or specifically binds to human CXCR3. Preferably, the antigen binding site binds to or specifically binds to a human CXCR3 molecule comprising, consisting essentially of or consisting of an amino acid sequence as shown in SEQ ID NO: 7

Preferably, the antigen binding site inhibits or reduces the CXCR3 activity induced by one or more ligands selected from the group consisting of CXCL9 (MIG), CXCL10 (IP10) and CXCL11 (I-TAC). For example, the antigen binding site may inhibit the migration of a cell, preferably an immune cell, stimulated by a CXCR3 ligand. Reduction or inhibition of CXCR3 activity may be determined by any method as described herein, particularly Example 4.

An antigen binding site as described herein may bind to CXCR3 and not detectably bind to or bind significantly to CXCR1, CXCR2, and/or C5aR. The binding of an antigen binding site to CXCR1, CXCR2, and/or C5aR may be determined by any method described herein, particularly flow cytometry as described in Example 2.

An antigen binding site as described herein may bind to CXCR3 and exhibit an ECso of less than 2 nM, less than 1 nM or less than 0.5 nM. Preferably the ECso of the antigen binding site is approximately 0.2 nM, or less. Preferably, the EC₅₀ is determined using a flow cytometry or ELISA assay as described herein, particularly Example 2.

An antigen binding site as described herein may exhibit an IC₅₀ in a competition binding assay with MIG, ITAC and/or IP10 of less than about 20, 15, 12, 10, 8, 6, 5, 4, 3, 2, or 1 nM.

An antigen binding site as described herein may inhibit the migration of an immune cell expressing CXCR3 at concentrations of 10 μg/ml, 1 μg/ml or less. Preferably, the migration assay is performed by any method as described herein, particularly Example 4.

An antigen binding site as described herein may bind to the first and/or second N-terminal loop in CXCR3. For example, the antigen binding site may bind to CXCR3 within residues 1 to 23 of the CXCR3 protein (numbering as per human CXCR3). Preferably, the antigen binding site of the invention binds to a peptide comprising the sequence: MVLEVSDHQVLNDAEVAALLENF (SEQ ID NO: 8), or a fragment thereof. Preferably the antigen binding site of the invention does not bind to a sequence of CXCR3 that is C-terminal to residue 23. Alternatively, the antigen binding site may bind to CXCR3 within residues 23 to 44 of the CXCR3 protein (numbering as per human CXCR3). Preferably, the antigen binding site of the invention binds to a peptide comprising the sequence: FSSSYDYGENESDSCCTSPPCP (SEQ ID NO: 10), or a fragment thereof.

The present invention also provides an antigen binding site which binds to an N-terminal region of CXCR3 and inhibits of CXCL9 (MIG), CXCL10 (IP10) and/or CXCL11 (I-TAC) binding to, or of CXCL9 (MIG), CXCL10 (IP10) and/or CXCL11 (I-TAC) mediated activity of, CXCR3. Preferably, the N-terminal region comprises, consists essentially of or consists of residues 1 to 23 (numbering as per human CXCR3). Preferably, the of CXCL9 (MIG), CXCL10 (IP10) and/or CXCL11 (I-TAC) mediated activity is of CXCL9 (MIG), CXCL10 (IP10) and/or CXCL11 (I-TAC) mediated chemotaxis of an immune cell (e.g. neutrophil).

As described herein, the antigen binding site may be in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3; or
(iv) one of (i) or (ii) linked to a protein that binds to an immune effector cell.

Further, as described herein, the antigen binding site may be in the form of:
(i) a diabody;
(ii) a triabody;
(iii) a tetrabody;
(iv) a Fab;
(v) a F(ab′)2;
(vi) a Fv;
(vii) one of (i) to (vi) linked to a constant region of an antibody, Fc or a heavy chain constant domain (CH) 2 and/or CH3; or
(viii) one of (i) to (vi) linked to a protein that binds to an immune effector cell.

The foregoing antigen binding sites can also be referred to as antigen binding domains of antibodies.

Preferably, an antigen binding site as described herein is an antibody or antigen binding fragment thereof. Typically, the antigen binding site is an antibody, for example, a monoclonal antibody.

As used herein the antigen binding site may be a variable domain.

In any aspect or embodiment, the antibody is a naked antibody. Specifically, the antibody is in a non-conjugated form and is not adapted to form a conjugate.

Reference herein to a protein or antibody that “binds to” CXCR3 provides literal support for a protein or antibody that “binds specifically to” or “specifically binds to” CXCR3.

The present invention also provides antigen binding domains or antigen binding fragments of the foregoing antibodies.

The invention also provides a fusion protein comprising an antigen binding site, immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody as described herein.

The invention also provides a conjugate in the form of an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody or fusion protein as described herein conjugated to a label or a cytotoxic agent.

The invention also provides an antibody for binding to an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein, or conjugate as described herein.

The invention also provides a nucleic acid encoding an antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

In one example, such a nucleic acid is included in an expression construct in which the nucleic acid is operably linked to a promoter. Such an expression construct can be in a vector, e.g., a plasmid.

In examples of the invention directed to single polypeptide chain antigen binding sites, the expression construct may comprise a promoter linked to a nucleic acid encoding that polypeptide chain.

In examples directed to multiple polypeptide chains that form an antigen binding site, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,
wherein the first and second polypeptides associate to form an antigen binding site as described herein.

Examples of cells of the present invention include bacterial cells, yeast cells, insect cells or mammalian cells.

The invention also provides a pharmaceutical composition comprising an antigen binding site, or comprising a CDR and/or FR sequence as described herein, or an immunoglobulin variable domain, antibody, dab (single domain antibody), di-scFv, scFv, Fab, Fab′, F(ab′)2, Fv fragment, diabody, triabody, tetrabody, linear antibody, single-chain antibody molecule, or multispecific antibody, fusion protein, or conjugate as described herein and a pharmaceutically acceptable carrier, diluent or excipient.

The invention also provides a diagnostic composition comprising an antigen binding site, or comprising a CDR and/or FR sequence as described herein, or antigen binding site, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein, a diluent and optionally a label.

The invention also provides a kit or article of manufacture comprising an antigen binding site, or comprising a CDR and/or FR sequence as described herein or an immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

An antigen binding site, a protein or antibody as described herein may comprise a human constant region, e.g., an IgG constant region, such as an IgG1, IgG2, or IgG3 or mixtures thereof. In the case of an antibody or protein comprising a VH and a VL, the VH can be linked to a heavy chain constant region and the VL can be linked to a light chain constant region.

In any aspect of the present invention, the antigen binding site comprises an Fc region that is engineered to have enhanced capacity to induce antibody-dependent cell-mediated cytotoxicity (ADCC). Preferably, the enhanced capacity to induce ADCC is conferred by mutation, deletion or modification of amino acids in the Fc region which interact with an Fc receptor. Preferably, the amino acids that are mutated, deleted or modified are at position 239, 330, and/or 332 as per SEQ ID NO:1 (where alanine is position 118) or at an equivalent position to 239, 330 and/or 332. Preferably, the amino acids are mutated to S239D, A330L and 1332E. Typically, the Fc comprises, consists essentially of or consists of an amino acid sequence shown in SEQ ID NO: 3.

In any aspect of the present invention, the antigen binding site comprises an Fc region that is not engineered to have a reduced capacity to induce antibody-dependent cell mediated cytotoxicity (ADCC). Preferably, there the amino acids at position 234, 235, and/or 331 as per SEQ ID NO: 1 (where alanine is position 118) or at an equivalent position to 234, 235 and/or 331 are not F, E and/or S respectively. In other words, the amino acid at position 234 is not F, at position 235 is not E and/or at 331 is not S.

In any aspect of the present invention, the antigen binding site does not comprise an Fc region comprising, consisting essentially of or consisting of an amino acid sequence as shown in SEQ ID NO: 2.

The functional characteristics of an antigen binding site as described herein will be taken to apply mutatis mutandis to an antibody of the invention.

An antigen binding site as described herein may be purified, substantially purified, isolated and/or recombinant.

An antigen binding site as described herein may be part of a supernatant taken from media in which a hybridoma expressing an antigen binding site as described herein has been grown.

In another aspect, the present invention also provides a method tor depleting CXCR3+ cells in a subject, comprising administering to the subject an effective amount of an antigen binding site as described herein.

The invention also provides a cell comprising a vector or nucleic acid molecule described herein.

The invention also provides an animal or tissue derived therefrom comprising a cell described herein.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 Long-lasting effect of CXCR3 depletion after a single IV injection. A depleting anti-CXCR3 antibody was injected into mice. CD4+, CD8+ and B cells were quantified at 24 and 36 days, post-injection. The results indicate that depletion of cells using a depleting anti-CXCR3 antibody is long-lasting.

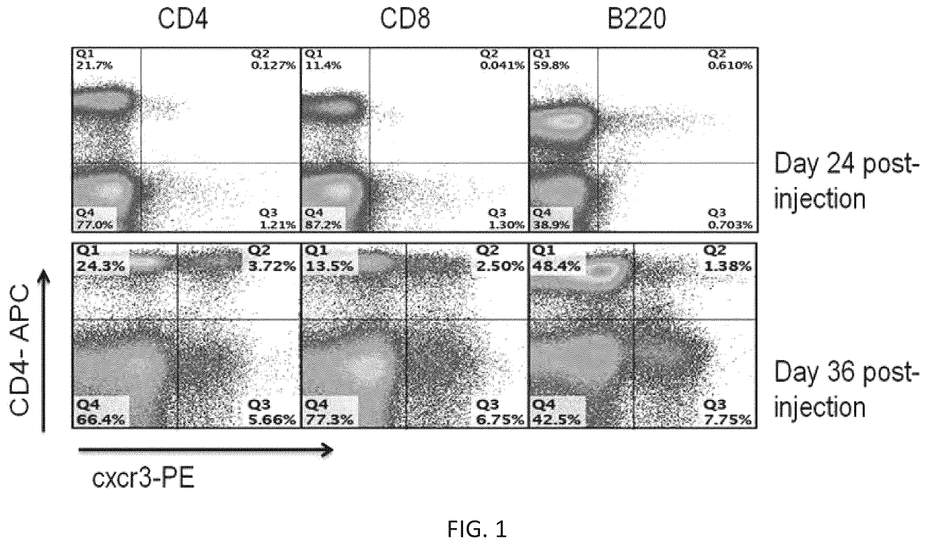

FIG. 2 Anti-CXCR3 depletion does not greatly affect Treg (CD4+; FoxP3+) numbers. A depleting anti-CXCR3 mAb mIgG1 (5 mg/kg) or m IgG1 isotype control (5 mg/kg) were injected IV in FoxP3-GFP reporter mice. After 4 days, CD4+ splenic lymphocyte populations were analyzed with anti-mCXCR3 from Biolegend. Those data confirm that only CXCRr3+ Treg subset were depleted but the ratio CD4+ FoxP3−/CD4+ FoxP3+ is unchanged.

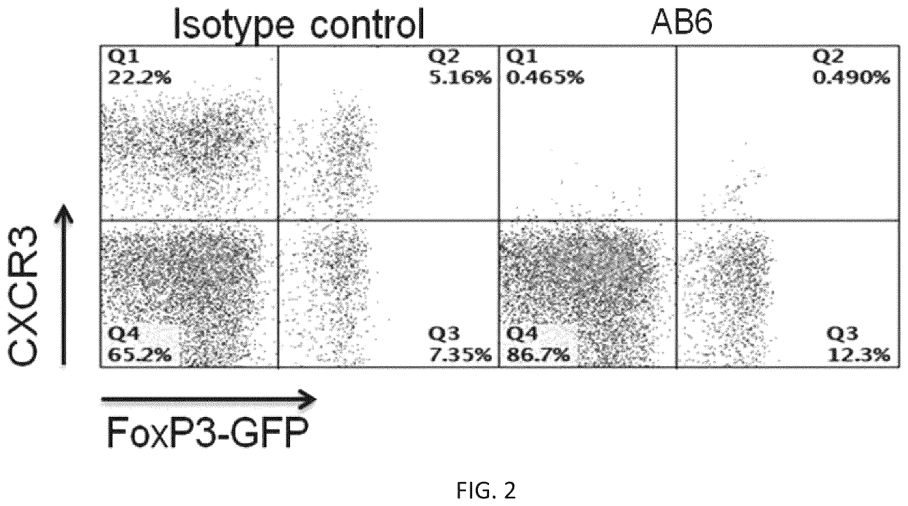

FIG. 3 Skin graft model. Skin transplantation experiment with depleting anti-CXCR3 antibody Balb/c to C57BL/6. Mice receiving depleting anti-CXCR3 antibody show evidence of accepted allografted skin, 200 days after transplantation.

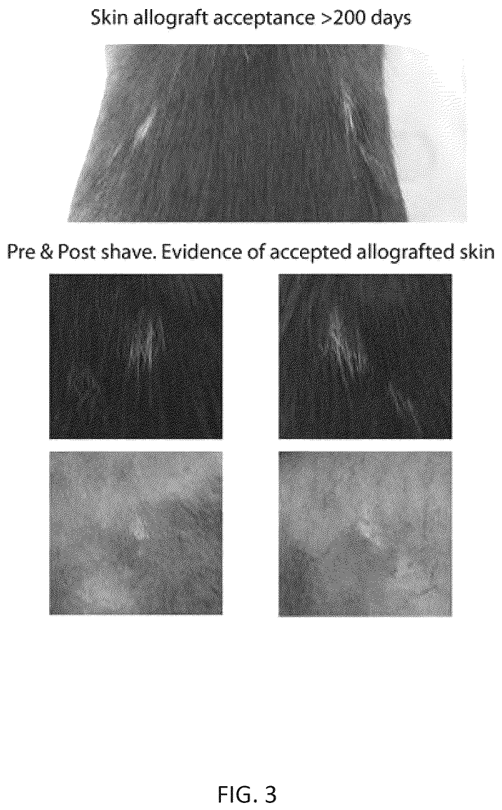

FIG. 4 Targeting CXCR3+ cells in NASH improves steatohepatitis and fibrosis. A depleting anti-CXCR3 mAb was shown to protect against NASH development in mice and to decrease collagen deposition (fibrosis).

FIG. 5 Depletion of CXCR3+ cells protects against NASH development. Mice were placed on a MCD diet in order to induce NASH. Mice also received either an isotype control; a depleting anti-CXCR3 mAb or a non-depleting anti-CXCR3 mAb. Only mice receiving the depleting antibody were protected from developing NASH.

FIG. 6 Anti-CXCR3 mAbs depletes NKT cells and activated intrahepatic CD8 T-cells. MCD diet increases the frequency of CD8+ CXCR3+ T-cells; MCD diet increase the frequency of NKT cells; NKT cells are CXCR3+ and are depleted after treatment with Fc engineered anti-CXCR3 mAB.

FIG. 7 Anti-CXCR3 treatment decreases the release of ALT in the serum and the infiltration of monocytes in the Liver.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have shown that various conditions can be treated using a antigen binding site that binds to CXCR3 and causes or mediates depletion of a cell expressing CXCR3. As a result, the methods described herein find particular application in the prevention or treatment of various conditions associated with CXCR3 expression, including, but not limited to, NASH.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only.

Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or AI-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### Selected Definitions

T cells are divided into several major subclasses, including cytotoxic T cells, which kill virus-infected cells, as well as two classes of regulatory cells, called helper T cells (Th cells) and suppressor T cells, which act to modulate the activity of other immune cells. During chronic infections, Th cells develop into at least three phenotypically and functionally distinct effector populations, Th1, Th2 and Th17 T cells. Th1 cells produce IFN-.gamma and IL-2, which are commonly associated with cell-mediated immune responses against various intracellular pathogens, whereas Th2 cells produce cytokines such as IL-4, IL-5, and IL-13, that are crucial to control extracellular helminthic infections. Th17 cells produce isoforms of IL-17, and Th17 cells play an important role in maintaining mucosal barriers and contributing to pathogen clearance at mucosal surfaces.

Th1 cells have been associated with organ-specific autoimmune diseases, delayed-type hypersensitivity, and transplant rejection. Further, cytokines such as IL-12 and IL-4 have dominant roles in determining the outcome of Th differentiation into Th1 and Th2 subsets, respectively. For example, in Th1 cells, following the binding of IL-12 to its cognate receptor, STAT4 is activated, thereby leading to the production of IFN-γ. Accordingly, STAT4-deficient mice are defective in Th1 differentiation and do not respond to intracellular pathogens such as Listeria monocytogenes.

Th1 cells express various chemokine receptors such as CXCR3, CCR1, and CCR5. Generally, CXCR3 is considered to be one of the best markers of Th1 cells, although CXCR3 is expressed also by NKT cells and some B cells, particularly those associated with inflammation (Qin et al J. Clin. Invest. 1997).

CXCR3 is also known as G protein-coupled receptor 9 (GPR9), CD182, CD183, CKR-L2, CMKAR3, GPR9, IP10-R, Mig-R, MigR). CXCR3 is an inflammatory chemoline receptor whose expression is associated with CDr+ Type-1 helper (Th1) and CD8+ cytotoxic lymphocytes (CTLs). In addition to CXCR3 expression on effector CD4+ and CD8+ T cells, CXCR3 is also highly expressed on innate lymphocytes, such as natural killer (NK) cells and N T cells, and on plasmacytoid DCs and subsets of B cells.

CXCR3 binds three chemokines: CXCL9 (also known as monokine induced by gammainterferon, or MIG), CXCL10 (interferon-induced protein of 10 kDa, or IP-10) and CXCL11 I (interferon-inducible T-cell alpha chemoattractant, or I-TAC). The binding of CXCR3 to these ligands, which themselves are induced by inflammatory cytokines, mediates cell migration, thereby coordinating inflammation in the periphery.

Because of the expression of CXCR3 on effector cells, and its role in cell migration and inflammation, there is a need for improved treatments to inhibit cell migration and/or inflammation. For example, migration of activated T-cells to the graft following transplantation results in donor organ damage and is a hallmark of acute and chronic rejection. A wide variety of immunosuppressive agents are available and routinely used in clinics to reduce the rejection episodes and to improve both short and long-term graft survival. However, their mode of action often results in a complete shut down of the immune system and their use relies on indefinite uptake. As such, they are typically associated with side effects such as opportunistic infections, lymphoproliferative disorders or cardiovascular diseases Accordingly, the development of more specific drugs able to induce transplantation tolerance and thus eliminate the constraint of high doses of continuous immunosuppressive agents is a major goal in the transplantation field.

Although IP-10 and MIG both belong to the CXC subfamily, in contrast to IL-8 and other CXC chemokines which are potent chemoattractants for neutrophils, the primary targets of IP-10 and MIG are lymphocytes, particularly effector cells such as activated or stimulated T lymphocytes and natural killer (NK) cells. Consistently, IP-10 and MIG lack the ELR motif, an essential binding epitope in those CXC chemokines that efficiently induce neutrophil chemotaxis. In addition, both recombinant human MIG and recombinant human IP-10 have been reported to induce calcium flux in tumor infiltrating lymphocytes (TIL). While IP-10 has been reported to induce chemotaxis of monocytes in vitro, the receptor responsible has not been identified, human MIG appears highly selective, and does not show such an effect. IP-10 expression is induced in a variety of tissues in inflammatory conditions such as psoriasis, fixed DRUG eruptions, cutaneous delayed-type hypersensitivity responses, tuberculoid leprosy, and in experimental glomerulonephritis, and experimental allergic encephalomyelitis. IP-10 also has a potent in vivo antitumor effect that is T cell dependent, is reported to be an inhibitor of angiogenesis in vivo, and can induce chemotaxis and degranulation of NK cells in vitro, suggesting a role as a mediator of NK cell recruitment and degranulation (in tumor cell destruction, for example). The expression patterns of IP-10 and MIG are also distinct in that expression of each is induced by interferon-gamma (IFNγ), while the expression of IL-8 is down-regulated by IFNγ.

Chemokines have been recently recognized as the long-sought mediators for the recruitment of lymphocytes. Several CXC chemokines were found to elicit lymphocyte chemotaxis but they are also active on granulocytes and monocytes. The situation is different for IP-10 and MIG, which are selective in their action on lymphocytes, including activated T lymphocytes and NK cells, and which bind CXCR3, a receptor which does not recognize numerous other chemokines and which displays a selective pattern of expression.

As used herein, reference to CXCR3 is to a molecule that has at least one biochemical or biophysical activity of CXCR3 as described herein. The term “CXCR3” as provided herein includes any of the C-X-C motif chemokine receptor 3 (CXCR3) protein naturally occurring forms, homologs or variants that maintain the activity of CXCR3 (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

For the purposes of nomenclature only and not a limitation, an exemplary amino acid sequence of human CXCR3 is SEQ ID NO: 7 and variants thereof having at least or about 85%, 90%, 91%, 92%, 93%, 94%, 95%, 96%, 97%, 98% or 99% sequence identity, including the natural variants set forth in SEQ ID NOs: 108 and 109, comprising the mutations R292Q and A363T, respectively. Exemplary non-human CXCR3 polypeptides include, but are not limited to, mouse (e.g. SEQ ID NO: 5), rat (e.g. SEQ ID NO: 8), pig (e.g. SEQ ID NO: 9). cow (e.g. SEQ ID NO: 10), rhesus macaque (e.g. SEQ ID NO: 11), and dog (e.g. SEQ ID NO: 12) CXCR3, and variants thereof having at least or about 85%, 90%, 91%, 92%, 93%, 94%, 95% 97%, 98% or 99% sequence identity thereto.

The phrase “inhibits CXCR3 activity” is understood to mean that the antigen binding site of the present invention inhibits or reduces any one or more activities of CXCR3, including but not limited to ligand binding to CXCR3; ligand induced conformational change of CXCR3; CXCR3 activation; G protein activation; CXCR3 mediated cell signalling; a CXCR3 mediated cell migratory, inflammatory, tumour growth, angiogenic or metastatic response in vitro or in vivo; CXCR3 mediated tumour cell growth; and/or CXCR3 mediated recruitment of lymphocytes.

“Inhibits MIG, IP-10 or I-TAC-mediated CXCR3 activity” is understood to mean that the antigen binding site of the present invention inhibits or reduces one or more activities described above that are mediated or induced by MIG, IP-10 and/or I-TAC. Further, the activity is measured using a suitable in vitro, cellular or in vivo assay and the activity is blocked or reduced by at least 1%, 5%, 10%, 25%, 50%, 60%, 70%, 80% or 90% or more, compared to CXCR3 activity in the same assay under the same conditions but without the antigen binding site. Preferably, the CXCR3 activity is mediated or induced by any one or more ligands, for example, MIG, IP-10 and I-TAC.

As used herein, “depletion” with respect to CXCR3+ cells (i.e. cells expressing CXCR3, preferably displaying cell surface CXCR3) refers to the reduction in activity or removal of these cells from a population of cells. Preferably, depletion with respect to CXCR3+ cells refers to removal or reduction in viability of these cells from a population of cells. Reference to depletion includes complete or partial depletion. Further, depletion may be permanent or temporary, and may be to varying extents in magnitude and/or spatially. Depletion may be the result of cell death, such as by apoptosis or necrosis. Preferably the cell death is mediated via ADCC. Alternatively, an antigen binding site per se as described herein may have little or no capacity to reduce the activity or viability of a CXCR3+ cell but it may be conjugated to or associated with a compound that has the capacity to reduce the activity or viability of a cell. Typically, an antigen binding site that has little or no capacity to reduce the activity or viability of a CXCR3+ cell is conjugated to or associated with a cytotoxic compound.

Depletion can be assessed by measuring the number of CXCR3+ cells in a population using any method known in the art (e.g. flow cytometry immunohistochemistry, etc.), before and after exposure to an antigen binding site, or antigen-binding fragment of the present invention, or in the absence and presence of an antigen binding site or antigen-binding fragment of the present invention. Following exposure to an antibody or antigen-binding fragment of the present invention, CXCR3+ cells can be depleted by at least or about 10%, 15%, 20%, 25%, 30%, 35%, 40%, 45%, 50%, 55%, 60%, 65%, 70%, 75%, 80%, 85%, 90%, 95% or more. An antigen binding site which has the capacity for depletion with respect to CXCR3+ cells may be referred to as a depleting antigen binding site.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding site” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens (e.g., CXCR3) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a K light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or AI-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding site or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding site as described herein reacts or associates more frequently, more rapidly, with greater duration and/or with greater affinity with a particular antigen or cell expressing same than it does with alternative antigens or cells. For example, an antigen binding site binds to CXCR3 (e.g., hCXCR3) with materially greater affinity (e.g., 1.5 fold or 2 fold or 5 fold or 10 fold or 20 fold or 40 fold or 60 fold or 80 fold to 100 fold or 150 fold or 200 fold) than it does to other CXCRs. In an example of the present invention, an antigen binding site that “specifically binds” to CXCR3 (preferably human) with an affinity at least 1.5 fold or 2 fold or greater (e.g., 5 fold or 10 fold or 20 fold r 50 fold or 100 fold or 200 fold) than it does to another chemokine receptor, such as CXCR1, CXCR2 and/or C5aR. Generally, but not necessarily, reference to binding means specific binding, and each term shall be understood to provide explicit support for the other term.

As used herein, the term “does not detectably bind” shall be understood to mean that an antigen binding site, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding site is immobilized and contacted with an antigen.

As used herein, the term “does not significantly bind” shall be understood to mean that the level of binding of an antigen binding site as described herein to a polypeptide is not statistically significantly higher than background, e.g., the level of binding signal detected in the absence of the antigen binding site and/or in the presence of a negative control protein (e.g., an isotype control antibody) and/or the level of binding detected in the presence of a negative control polypeptide. The level of binding is detected using biosensor analysis (e.g. Biacore) in which the antigen binding site is immobilized and contacted with an antigen.

As used herein, the term “epitope” (syn. “antigenic determinant”) shall be understood to mean a region of CXCR3 to which an antigen binding site comprising an antigen binding domain of an antibody binds. Unless otherwise defined, this term is not necessarily limited to the specific residues or structure to which the antigen binding site makes contact. For example, this term includes the region spanning amino acids contacted by the antigen binding site and 5-10 (or more) or 2-5 or 1-3 amino acids outside of this region. In some examples, the epitope comprises a series of discontinuous amino acids that are positioned close to one another when antigen binding site is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “condition” refers to a disruption of or interference with normal function, and is not to be limited to any specific condition, and will include diseases or disorders.

As used herein, the terms “preventing”, “prevent” or “prevention” include administering an antigen binding site as described herein to thereby stop or hinder the development of at least one symptom of a condition. This term also encompasses treatment of a subject in remission to prevent or hinder relapse.

As used herein, the terms “treating”, “treat” or “treatment” include administering an antigen binding site described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

### Antibodies

In one example, an antigen binding site or CXCR3-binding protein as described herein according to any example is an antibody.

Anti-CXCR3 antibodies: various anti-CXCR3 antibodies are commercially available, including but not limited to: 1C6 (anti-human CXCR3) or clone 173 (anti-mouse CXCR3), both available from Biolegend, BDBiosciences, AbCam or other supplier. Alternatively, the antibody may be an antibody as described in WO2018/119299.

Methods for generating antibodies are known in the art and/or described in Harlow and Lane (editors) Antibodies: A Laboratory Manual, Cold Spring Harbor Laboratory, (1988). Generally, in such methods CXCR3 (e.g., hCXCR3) or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present invention may be a synthetic antibody. For example, the antibody is a chimeric antibody, a humanized antibody, a human antibody synhumanized antibody, primatized antibody or a de-immunized antibody.

### Antibody Binding Domain Containing Proteins

### Single-Domain Antibodies

In some examples, a protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, MA; see, e.g., U.S. Pat. No. 6,248,516).

### Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “VHH domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Proteins Comprising Antigen Binding Domains Thereof

The present invention also contemplates other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab3 (e.g., as described in EP19930302894).

### Mutations to Proteins

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding site described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding site as described herein comprising one or more conservative amino acid substitutions compared to a sequence set forth herein. In some examples, the antigen binding site comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), #-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding site comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding site as described herein. In another example, the mutation(s) occur within a CDR of an antigen binding site as described herein.

Exemplary methods for producing mutant forms of an antigen binding site include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1 Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant antigen binding sites as described herein will be apparent to the skilled artisan and/or described herein, e.g., antigen binding. For example, methods for determining antigen binding, competitive inhibition of binding, affinity, association, dissociation and therapeutic efficacy are described herein.

### Constant Regions

The present invention encompasses antigen binding sites and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2 and IgG3.

In one example, the Fc region of the constant region has an enhanced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In any aspect of the present invention, the antigen binding site comprises an Fc region that is engineered to have enhanced capacity to induce antibody-dependent cell-mediated cytotoxicity (ADCC). Preferably, the enhanced capacity to induce ADCC is conferred by mutation, deletion or modification of amino acids in the Fc region which interact with an Fc receptor. Preferably, the amino acids that are mutated, deleted or modified are at position 239, 330, and/or 332 as per SEQ ID NO:1 (where alanine is position 118) or at an equivalent position to 239, 330 and/or 332. Preferably, the amino acids are mutated to S239D, A330L and 1332E. Typically, the Fc comprises, consists essentially of or consists of an amino acid sequence shown in SEQ ID NO: 3.

In any aspect of the present invention, the antigen binding site comprises an Fc region that is not engineered to have a reduced capacity to induce antibody-dependent cell mediated cytotoxicity (ADCC). Preferably, there the amino acids at position 234, 235, and/or 331 as per SEQ ID NO: 1 (where alanine is position 118) or at an equivalent position to 234, 235 and/or 331 are not F, E and/or S respectively. In other words, the amino acid at position 234 is not F, at position 235 is not E and/or at 331 is not S.

In any aspect of the present invention, the antigen binding site does not comprise an Fc region comprising, consisting essentially of or consisting of an amino acid sequence as shown in SEQ ID NO: 2.

In any aspect of the present invention, the antigen binding site per se as described herein may have little or no capacity to reduce the activity or viability of a CXCR3+ cell but it may be conjugated to or associated with a compound that has the capacity to reduce the activity or viability of a cell. For example, the Fc region may have a reduced ability or capacity to induce effector function, e.g., compared to a native or wild-type human IgG1 or IgG3 Fc region, however in this circumstance, the antigen binding site is conjugated to or associated with a cytotoxic compound.

One example of an Fc region with reduced effector function is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}³ domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

In any of these examples, the antigen binding site that has reduced effector function is conjugate to or associated with a compound that can reduce the activity or viability of a cell.

### Additional Modifications

The present invention also contemplates additional modifications to an antibody or antigen binding site comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

### Protein Production

In one example, an antigen binding site described herein according to any example is produced by culturing a hybridoma under conditions sufficient to produce the protein, e.g., as described herein and/or as is known in the art.

### Recombinant Expression

In another example, an antigen binding site described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the not promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FIO (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where an antigen binding site is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding site prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Assaying Activity of an Antigen Binding Site

### Binding to CXCR3 and Mutants Thereof

It will be apparent to the skilled artisan from the disclosure herein that antigen binding sites described herein bind to CXCR3. Methods for assessing binding to a protein are known in the art, e.g., as described in Scopes (In: Protein purification: principles and practice, Third Edition, Springer Verlag, 1994). Such a method generally involves immobilizing the antigen binding site and contacting it with labeled antigen (CXCR3). Following washing to remove non-specific bound protein, the amount of label and, as a consequence, bound antigen is detected. Of course, the antigen binding site can be labeled and the antigen immobilized. Panning-type assays can also be used. Alternatively, or additionally, surface plasmon resonance assays can be used.

Optionally, the dissociation constant (Kd), association constant (Ka) and/or affinity constant (K_{D}) of an immobilized antigen binding site for CXCR3 or an epitope thereof is determined. The “Kd” or “Ka” or “K_{D}” for an CXCR3-binding protein is in one example measured by a radiolabeled or fluorescently-labeled CXCR3 ligand binding assay. In the case of a “Kd”, this assay equilibrates the antigen binding site with a minimal concentration of labeled CXCR3 or epitope thereof in the presence of a titration series of unlabeled CXCR3. Following washing to remove unbound CXCR3 or epitope thereof, the amount of label is determined, which is indicative of the Kd of the protein.

According to another example the Kd, Ka or K_{D} is measured by using surface plasmon resonance assays, e.g., using BIAcore surface plasmon resonance (BIAcore, Inc., Piscataway, NJ) with immobilized CXCR3 or a region thereof or immobilized antigen binding site.

### Determining Inhibitory Activity

In some examples of the present invention, a protein is capable of inhibiting CXCR3 activity.

Various assays are known in the art for assessing the ability of a protein to inhibit or reduce signaling of a ligand through a receptor leading to a functional response.

In one example, the antigen binding site inhibits migration of immune cells (e.g., neutrophil cells) expressing CXCR3 which are cultured in the presence of a CXCR3 ligand (e.g. MIG, IP-10 and I-TAC). Methods for assessing migration are known in the art and described herein. An antigen binding site that inhibits migration compared to the level observed in the absence of the antigen binding site is considered to inhibit or reduce CXCR3 activity, specifically CXCR3 mediated signalling and migration. Other assays to determine inhibitory activity of the anti-CXCR3 antibodies include calcium flux assay, radioligand binding assay, cAMP assay and beta arrestin recruitment assay. Any of the assays described herein may include one or more CXCR3 ligands including MIG, IP-10 and I-TAC.

### Conditions to be Treated

The antigen binding sites of the present invention are useful in the treatment or prevention of any condition associated, or caused by, the presence or over-expression of CXCR3.

The antigen binding sites of the present invention are useful in the treatment or prevention of any condition associated, or caused by, Th1 cells.

The antigen binding sites of the present invention are useful in the treatment or prevention of a condition associated, or caused by acute or chronic inflammation such as fibrosis, fatty liver disease, including NASH and transplant rejection (graft versus host disease, GVHD). The antigen binding sites of the invention preferably find application in the treatment or prevention of autoimmune inflammatory conditions.

In still further embodiments, the antigen binding site of the invention may find application in the reduction of fibrosis associated with an autoimmune disease characterised by inflammation.

“Fibrosis”, “Fibrotic disease” or “Fibro proliferative disease” means the formation of excess fibrous connective tissue in a reparative process upon injury. Scarring is a result of continuous fibrosis that obliterates the affected organs or tissues architecture. As a result of abnormal reparative processes, which do not clear the formed scar tissue, fibrosis progresses further. Fibrosis can be found in various tissues, including the heart, the lungs, the liver, the skin, blood vessels and the kidneys. Examples of fibrosis are described herein and include pulmonary fibrosis, liver cirrhosis, systemic sclerosis, progressive kidney disease and cardiac fibrosis associated with various cardiovascular diseases.

A subject may be identified as having fibrosis by determining if a subject has organ dysfunction, scarring, alteration of normal extracellular matrix balance, increase in collagen deposition, increased collagen volume fraction, differentiation of fibroblasts to myofibroblasts, reduction in the level of matrix metalloproteinases and increase in the level of tissue Inhibitors of matrix metalloproteinases, increased levels of either N-terminal or C-terminal propeptide of type I procollagen (PINP or PICP) and decreased levels of C-terminal telopeptide of Type I Collagen (CTP or CITP), increased collagen deposition and impaired cardiac function measured by various noninvasive imaging techniques, impaired renal function measured by increased proteinurea and albuminurea, decreased glomerular filtration rate, doubling of plasma creatinine levels.

According to a preferred embodiment of the invention, the liver fibrosis is resulting from a chronic liver disease, hepatitis B virus infection, hepatitis C virus infection, hepatitis D virus infection, schistosomiasis, alcoholic liver disease or non-alcoholic steatohepatitis, non-alcoholic fatty liver disease, obesity, diabetes, protein malnutrition, coronary artery disease, auto-immune hepatitis, cystic fibrosis, alpha-1—antitrypsin deficiency, primary biliary cirrhosis, drug reaction and exposure to toxins.

The term “liver fibrosis” means the formation or development of excess fibrous connective tissue (fibrosis) in the liver thereby resulting in the development of scarred (fibrotic) tissue. The scarred tissue replaces healthy tissue by the process of fibrosis and leads to subsequent cirrhosis of the liver. Liver fibrosis may be associated with non-alcoholic steatohepatitis (NASH).

The liver fibrosis may include, but not be limited to, cirrhosis, and associated conditions such as chronic viral hepatitis, non-alcoholic fatty liver disease (NAFLD), alcoholic steatohepatitis (ASH), non-alcoholic steatohepatitis (NASH), primary biliary cirrhosis (PBC), biliary cirrhosis, autoimmune hepatitis). Lung fibrosis may include idiopathic pulmonary fibrosis (IPF) or cryptogenic fibrosing alveolitis, chronic fibrosing interstitial pneumonia, interstitial lung disease (ILD), and diffuse parenchymal lung disease (DPLD)). Cardiac fibrosis, congestive heart failure, cardiomyopathy, post-myocardial infarction defects in heart function; peripheral vascular disease; rheumatoid arthritis; glaucoma; age-related macular degeneration (wet AMD and dry AMD); emphysema, chronic obstructive pulmonary disease (COPD); multiple sclerosis; and chronic asthma may also be prevented, treated, or ameliorated with compositions, methods or uses as described herein.

### Fatty Liver Disease and NASH

The present invention also relates to methods for preventing the onset of, preventing the progression of, or treating fatty liver disease in an individual who is at risk of developing fatty liver disease or is displaying symptoms of fatty liver disease.

In certain preferred embodiments, the present invention provides methods for preventing the onset of, preventing the progression of, or treating NASH in an individual who is at risk of developing NASH or is displaying symptoms of NASH.

Fatty liver disease that develops in the absence of alcohol abuse is recognized increasingly as a major health burden, in particular if an inflammatory component is involved such as in non-alcoholic steatohepatitis (NASH). Estimates based on imaging and biopsy studies suggest that about 20% to 30% of adults in the United States and other Western countries have excess fat accumulation in the liver. About 10% of these individuals, or fully 2% to 3% of adults, are estimated to meet current diagnostic criteria for NASH. Sustained liver injury leads to progressive fibrosis and cirrhosis in about 30% of NASH patients. The diagnostic criteria for NASH continue to evolve and rely on the histologic findings of steatosis, hepatocellular injury (ballooning, Mallory bodies), and the pattern of fibrosis.

As used herein, NAFLD refers to non-alcoholic fatty liver disease. NAFLD, which is considered to be a non-life threatening to disease, is to be distinguished from NASH which is a more severe form of NAFLD, and associated with increased mortality. The terms “non-alcoholic fatty liver disease” or “NAFLD”, “non-alcoholic steatohepatitis” or “NASH”, and “Simple Steatosis” are each used in the sense which are currently admitted by the scientific community. In general, NAFLD exists as a histological spectrum of changes. All of the stages of NAFLD have in common the accumulation of fat in the liver cells (steatosis). Simple steatosis refers to the hepatic steatosis in the absence of significant inflammation and hepatocellular damage, whereas NASH demonstrates inflammation and hepatocellular damage and sometimes fibrosis.

The skilled person will be familiar with methods for determining if an individual has, or is at risk of developing non-alcoholic steatohepatitis (NASH). In particular, the skilled person will be familiar with methods for determining if an individual has symptoms or signs of non-alcoholic fatty liver disease, including whether the individual has, or is at risk of any of the subtypes of fatty liver disease such as steatosis, NASH or NAFDL, and, in particular, for the differentiation of the life threatening NASH from the less severe NAFLD.

The term “fatty liver disease” is well known in the art. Preferably, the term refers to an impairment of the liver. Preferably, said impairment is the result of a surplus of triacylglyceride that accumulate in the liver and form large vacuoles. The symptoms accompanying fatty liver disease are well known from standard text books of medicine such as Stedman's or Pschyrembel. Fatty liver disease may result from alcohol abuse, diabetes mellitus, nutritional defects and wrong diets, toxicity of drugs or genetic predisposition. Fatty liver disease as used in accordance with the present invention also include the more severe forms thereof and, in particular, steatosis, NASH or NAFDL. Symptoms accompanying these diseases are also well known to the physicians and are described in detail in standard text books of medicine.

Liver biopsy has remained the criterion standard or “gold standard” in the evaluation of the etiology and extent of disease of the liver such as NAFLD and NASH. Percutaneous liver biopsy is the preferred method to determine NAFLD and to differentiate NASH from NAFLD. Other biopsy methods are typically even more invasive and include transvenous and laparoscopic liver biopsy. The American Gastroenterological Association has published detailed recommendations on how to grade NAFLD comprising NASH into macrovescicular steatosis grades, necroinflammatory activity grades and fibrosis stages (American Gastroenterological Association 2002, Gastroenterology 123: 1705-25; Brunt 1999, Am J Gastroenterol. 94: 2467-74, Brunt 2010, Nat Rev Gastroenterol Hepatol. 7:195-203).

While simple steatosis appears to be a relatively benign condition, it may progress to NASH over time. Accordingly, a person at risk of NASH may be a person diagnosed as having simple steatosis. In addition to its association with cardiovascular complications, NAFLD can lead to liver related morbidity and mortality. The risk of developing cirrhosis is higher in the presence of NASH, which is more likely in the presence of the following features:
type 2 diabetes mellitus (T2DM)
obesity (body mass index [BMI]>30 kg/m2)
age more than 50 years
serum aminotransferases (ALT or AST) more than two times the upper limit of normal.

Accordingly, a person may be considered to be at risk of NASH if they are diagnosed with NAFLD and have one or more of the above features. A definitive diagnosis of NAFLD depends on three factors:
1) evidence of fatty infiltration from either imaging (ultrasound, magnetic resonance imaging [MRI]) or histology (liver biopsy)
2) exclusion of significant alcohol consumption
3) exclusion of other causes of hepatic steatosis (eg. medications, surgery, metabolic disorders).

In addition to the above, signs of cirrhotic complications may also be considered, including signs of portal hypertension (splenomegaly, increased portal vein size, varices) or other complications such as HCC, portal vein thrombosis, or ascites. The risk of fibrosis and progressive liver disease in NAFLD increases with severity of insulin resistance. The number of metabolic syndrome features (obesity, combined hyperlipidemia, diabetes mellitus (type II), and high blood pressure) can be used to estimate risk of insulin resistance. The presence of three or more features of the syndrome, especially if these include central adiposity and type 2 diabetes mellitus (T2DM) are predictive of the presence of NASH rather than simple steatosis. In addition, family history plays a role: an individual with a first degree relative with T2DM has a 90% chance of developing T2DM, and therefore NASH. Central adiposity can be assessed using waist circumference measured at the narrowest point mid-way between the lowest rib and the iliac crest at the end of expiration with the patient standing.

Non-invasive tools for estimating the degree of fibrosis include transient elastography (FibroScan®), acoustic radiation force impulse (ARFI), and non-invasive biomarker algorithms such as NAFLD Fibrosis Score, FibroTest and Hepascore.

The skilled person will also be familiar with methods for determining whether an individual has been successfully treated so as to prevent the further progression of NASH, reversal of symptoms of NASH or prevention of the development of NASH in an at-risk individual. Successful treatment or prevention can be determined by measuring for an improvement, or reduction in the rate of progression or worsening of any one or more of the symptoms described above associated with NASH.

### Transplant Rejection (Graft Versus Host Disease)

The methods of the present invention also have utility in treating or preventing the onset or progression of GVHD, or reducing the severity of GVHD in an individual requiring cell transplantation.

GVHD is an inflammatory disease initiated by T cells in the donor graft that recognize histocompatibility and other tissue antigens of the host and GVHD is mediated by a variety of effector cells and inflammatory cytokines. GVHD presents in both acute and chronic forms. The most common symptomatic organs are the skin, liver, and gastrointestinal tract, including the oral cavity and oropharyngeal regions. GVHD may involve other organs such as the lung.

Treatment of GVHD is generally only 50-75% successful; the remainder of patients generally do not survive. The risk and severity of this immune-mediated condition are directly related to the degree of mismatch between a host and the donor of hematopoietic cells. For example, GVHD develops in up to 30% of recipients of human leukocyte antigen (HLA)-matched sibling marrow, in up to 60% of recipients of HLA-matched unrelated donor marrow, and in a higher percentage of recipient of HLA-mismatched marrow. Patients with mild intestinal GVHD present with anorexia, nausea, vomiting, abdominal pain and diarrhea, whereas patients with severe GVHD are disabled by these symptoms. If untreated, symptoms of intestinal GVHD persist and often progress; spontaneous remissions are unusual. In its most severe form, GVHD leads to necrosis and exfoliation of most of the epithelial cells of the intestinal mucosa, a frequently fatal condition. The symptoms of acute GVHD usually present within 100 days of transplantation. The symptoms of chronic GVHD usually present somewhat later, up to three years after allogeneic HCT, and are often proceeded by a history of acute GVHD.

Two distinct types of GVHD are clinically recognized, acute and chronic. The acute form of the disease usually develops within the first three months after transplantation. The incidence rate of acute GVHD is estimated at 30-50% among patients receiving transplant from HLA-identical sibling donors, and 50-70% in patients receiving HLA-matched unrelated transplants. Severe acute GVHD (grade III-IV) occurs in up to 20% of recipients of related donors and up to 35% of unrelated donors. Severe acute GVHD carries a poor prognosis, with 25% long term survival for grade Ill and 5% for grade IV.

The skilled person will be able to identify an individual in need for treatment or prevention of GVHD, including by identifying patient groups most likely to receive cell transplants and patients at risk of acute or showing signs of chronic GVHD. For example, chronic GVHD occurs in up to 60% of patients receiving HLA-identical sibling marrow grafts and 70% of patients receiving alternative donor marrow grafts who survive beyond day 100. Symptoms of chronic GVHD usually present between 3 months and 2 years after allogeneic transplantation, and about two thirds develop within the first 12 months. Altogether, only less than 20% of transplanted patients do not develop either acute or chronic GVHD.

### Compositions

In some examples, an antigen binding site as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding site into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding site dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding site of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, an antigen binding site as described herein will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding site of the present invention.

WO2002/080967 describes compositions and methods for administering aerosolized compositions comprising antibodies for the treatment of, e.g., asthma, which are also suitable for administration of an antigen binding site of the present invention.

### Dosages and Timing of Administration

Suitable dosages of an antigen binding site of the present invention will vary depending on the specific an antigen binding site, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding site(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding site(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding site(s), rather the present invention encompasses any amount of the antigen binding site(s) sufficient to achieve the stated result in a subject.

### Kits

The present invention additionally comprises a kit comprising one or more of the following:
(i) an antigen binding site as described herein or expression construct(s) encoding same;
(ii) a cell of the invention;
(iii) a complex of the invention; or
(iii) a pharmaceutical composition of the invention.

In the case of a kit for detecting CXCR3, the kit can additionally comprise a detection means, e.g., linked to a antigen binding site of the invention.

In the case of a kit for therapeutic/prophylactic use, the kit can additionally comprise a pharmaceutically acceptable carrier.

Optionally a kit of the invention is packaged with instructions for use in a method described herein according to any example.

TABLE 1
Summary of amino acid and nucleotide sequences

| Antibody | | SEQ ID | |
|---|---|---|---|
| ID | Region | NO: | Amino acid or nucleotide sequence |
| hIgG1 | Fc region | 1 | A S T K G P S V F P L A P S S K S T S G G T A A L |
| | | | G C L V K D Y F P E P V T V S W N S G A L T S G V |
| | | | H T F P A V L Q S S G L Y S L S S V V T V P S S S |
| | | | L G T Q T Y I C N V N H K P S N T K V D K K V E P |
| | | | K S C D K T H T C P P C P A P E L L G G P S V F L |
| | | | F P P K P K D T L M I S R T P E V T C V V V D V S |
| | | | H E D P E V K F N W Y V D G V E V H N A K T K P R |
| | | | E E Q Y N S T Y R V V S V L T V L H Q D W L N G K |
| | | | E Y K C K V S N K A L P A P I E K T I S K A K G Q |
| | | | P R E P Q V Y T L P P S R D E L T K N Q V S L T C |
| | | | L V K G F Y P S D I A V E W E S N G Q P E N N Y K |
| | | | T T P P V L D S D G S F F L Y S K L T V D K S R W |
| | | | Q Q G N V F S C S V M H E A L H N H Y T Q K S L S |
| | | | L S P G K (bold is CH1, italics hinge region, |
| | | | underlined is CH2 and no formatting is CH3)) |
| 3SFc | Fc region | 2 | A S T K G P S V F P L A P S S K S T S G G T A A L |
| | | | G C L V K D Y F P E P V T V S W N S G A L T S G V |
| | | | H T F P A V L Q S S G L Y S L S S V V T V P S S S |
| | | | L G T Q T Y I C N V N H K P S N T K V D K K V E P |
| | | | K S C D K T H T C P P C P A P E F E G G P S V F L |
| | | | F P P K P K D T L M I S R T P E V T C V V V D V S |
| | | | H E D P E V K F N W Y V D G V E V H N A K T K P R |
| | | | E E Q Y N S T Y R V V S V L T V L H Q D W L N G K |
| | | | E Y K C K V S N K A L P A S I E K T I S K A K G Q |
| | | | P R E P Q V Y T L P P S R D E L T K N Q V S L T C |
| | | | L V K G F Y P S D I A V E W E S N G Q P E N N Y K |
| | | | T T P P V L D S D G S F F L Y S K L T V D K S R W |
| | | | Q Q G N V F S C S V M H E A L H N H Y T Q K S L S |
| | | | L S P G K |
| 3MFc | Fc region | 3 | A S T K G P S V F P L A P S S K S T S G G T A A L |
| | | | G C L V K D Y F P E P V T V S W N S G A L T S G V |
| | | | H T F P A V L Q S S G L Y S L S S V V T V P S S S |
| | | | L G T Q T Y I C N V N H K P S N T K V D K K V E P |
| | | | K S C D K T H T C P P C P A P E L L G G P D V F L |
| | | | F P P K P K D T L M I S R T P E V T C V V V D V S |
| | | | H E D P E V K F N W Y V D G V E V H N A K T K P R |
| | | | E E Q Y N S T Y R V V S V L T V L H Q D W L N G K |
| | | | E Y K C K V S N K A L P L P E E K T I S K A K G Q |
| | | | P R E P Q V Y T L P P S R D E L T K N Q V S L T C |
| | | | L V K G F Y P S D I A V E W E S N G Q P E N N Y K |
| | | | T T P P V L D S D G S F F L Y S K L T V D K S R W |
| | | | Q Q G N V F S C S V M H E A L H N H Y T Q K S L S |
| | | | L S P G K |
| mCXCR3 | DNA | 4 | atgtaccttgaggttagtgaacgtcaagtgctagatgcctcggactttgc |
| | | | ctttcttctggaaaacagca cctctccctacgattatggg gaaaacgaga |
| | | | gcgacttctctgactccccgccctgcccacaggatttcagcctgaacttt |
| | | | gacagaaccttcctgccagccctctacagcctcctcttcttgctggggct |
| | | | gctaggcaatggggcggtggctgctgtgctactgagtcagcgcactgccc |
| | | | tgagcagcacggacaccttcctgctccacctggctgtagccgatgttctg |
| | | | ctggtgttaactcttccattgtgggcagtggatgctgctgtccagtgggt |
| | | | tttcggccctggcctctgcaaagtggcaggcgccttgttcaacatcaact |
| | | | tctatgcaggggccttcctgctggcttgtataagcttcgacagatatctg |
| | | | agcatagtgcacgccacccagatctaccgcagggacccccgggtacgtgt |
| | | | agccctcacctgcatagttgtatggggtctctgtctgctctttgccctcc |
| | | | cagatttcatctacctatcagccaactacgatcagcgcctcaatgccacc |
| | | | cattgccagtacaacttcccacaggtgggtcgcactgctc tgcgtgtact |
| | | | gcagctagtggctggtttcctgctgccccttctggtcatggcctactgct |
| | | | atgcccatatcctagctgttctgctggtctccagaggccagaggcgtttt |
| | | | cgagctatgaggctagtggtagtggtggtggcagcctttgctgtctgctg |
| | | | gaccccctatcacctggtggtgctagtggatatcctcatggatgtgggag |
| | | | ttttggcccgcaactgtggtcgagaaagccacgtggatgtggccaagtca |
| | | | gtcacctcgggcatggggtacatgcactgctgcctcaatccgctgctcta |
| | | | tgcctttgtgggagtgaagttcagagagcaaatgtggatgttgttcacgc |
| | | | gcctgggccgctctgaccagagagggccccagcggcagccgtcatcttca |
| | | | cggagagaatcatcctggtctgagacaactgaggcctcctacctgggcttg |
| mCXCR3 | Protein | 5 | MYLEVSERQVLDASDFAFLL |
| | | | ENSTSPYDYGENESDFSDSP |
| | | | PCPQDFSLNFDRTFLPALYS |
| | | | LLFLLGLLGNGAVAAVLLSQ |
| | | | RTALSSTDTFLLHLAVADVL |
| | | | LVLTLPLWAVDAAVQWVFGP |
| | | | GLCKVAGALFNINFYAGAFL |
| | | | LACISFDRYLSIVHATQIYR |
| | | | RDPRVRVALTCIVVWGLCLL |
| | | | FALPDFIYLSANYDQRLNAT |
| | | | HCQYNFPQVGRTALRVLQLV |
| | | | AGFLLPLLVMAYCYAHILAV |
| | | | LLVSRGQRRFRAMRLVVVVV |
| | | | AAFAVCWTPYHLVVLVDILM |
| | | | DVGVLARNCGRESHVDVAKS |
| | | | VTSGMGYMHCCLNPLLYAFV |
| | | | GVKFREQMWMLFTRLGRSDQ |
| | | | RGPQRQPSSSRRESSWSETT |
| | | | EASYLGL |
| hCXCR3 | DNA | 6 | ccaaccacaagcaccaaagcagaggggcaggcagcacaccacccag |
| | | | cagccagagcaccagcccagccatggtccttgaggtgagtgaccaccaa |
| | | | gtgctaaatgacgccgaggttgccgccctcctggagaacttcagctottccta |
| | | | tgactatggagaaaacgagagtgactcgtgctgtacctccccgccctgccc |
| | | | acaggacttcagcctgaacttcgaccgggccttcctgccagccctctacag |
| | | | cctcctctttctgctggggctgctgggcaacggcgcggtggcagccgtgctg |
| | | | ctgagccggcggacagccctgagcagcaccgacaccttcctgctccacct |
| | | | agctgtagcagacacgctgctggtgctgacactgccgctctgggcagtgga |
| | | | cgctgccgtccagtgggtctttggctctggcctctgcaaagtggcaggtgccc |
| | | | tcttcaacatcaacttctacgcaggagccctcctgctggcctgcatcagctttg |
| | | | accgctacctgaacatagttcatgccacccagctctaccgccgggggcccc |
| | | | cggcccgcgtgaccctcacctgcctggctgtctgggggctctgcctgcttttc |
| | | | gccctcccagacttcatcttcctgtcggcccaccacgacgagcgcctcaac |
| | | | gccacccactgccaatacaacttcccacaggtgggccgcacggctctgcg |
| | | | ggtgctgcagctggtggctggctttctgctgcccctgctggtcatggcctactg |
| | | | ctatgcccacatcctggccgtgctgctggtttccaggggccagcggcgcctg |
| | | | cgggccatgcggctggtggtggtggtcgtggtggcctttgccctctgctggac |
| | | | cccctatcacctggtggtgctggtggacatcctcatggacctgggcgctttgg |
| | | | cccgcaactgtggccgagaaagcagggtagacgtggccaagtcggtcac |
| | | | ctcaggcctgggctacatgcactgctgcctcaacccgctgctctatgcctttgt |
| | | | aggggtcaagttccgggagcggatgtggatgctgctcttgcgcctgggctgc |
| | | | cccaaccagagagggctccagaggcagccatcgtcttcccgccgggattc |
| | | | atcctggtctgagacctcagaggcctcctactcgggcttgtgaggccggaat |
| | | | ccgggctcccctttcgcccacagtctgacttccccgcattccaggctcctccct |
| | | | ccctctgccggctctggctctccccaatatcctcgctcccgggactcactggc |
| | | | agccccagcaccaccaggtctcccgggaagccaccctcccagctctgag |
| | | | gactgcaccattgctgctccttagctgccaagccccatcctgccgcccgagg |
| | | | tggctgcctggagccccactgcccttctcatttggaaactaaaacttcatcttc |
| | | | cccaagtgcggggagtacaaggcatggcgtagagggtgctgccccatga |
| | | | agccacagcccaggcctccagctcagcagtgactgtggccatggtcccca |
| | | | agacctctatatttgctcttttatttttatgtctaaaatcctgcttaaaacttt |
| | | | tcaataaacaagatcgtcaggaccaaaaaaaaaaaaaaaaaaaaaaaaaaa |
| | | | aaaaaaaaaaaaaaaaaaaa |
| hCXCR3 | Protein | 7 | MVLEVSDHQVLNDAEVAALL |
| | | | ENFSSSYDYGENESDSCCTS |
| | | | PPCPQDFSLNFDRAFLPALY |
| | | | SLLFLLGLLGNGAVAAVLLS |
| | | | RRTALSSTDTFLLHLAVADT |
| | | | LLVLTLPLWAVDAAVQWVFG |
| | | | SGLCKVAGALFNINFYAGAL |
| | | | LLACISFDRYLNIVHATQLY |
| | | | RRGPPARVTLTCLAVWGLCL |
| | | | LFALPDFIFLSAHHDERLNA |
| | | | THCQYNFPQVGRTALRVLQL |
| | | | VAGFLLPLLVMAYCYAHILA |
| | | | VLLVSRGQRRLRAMRLVVVV |
| | | | VVAFALCWTPYHLVVLVDIL |
| | | | MDLGALARNCGRESRVDVAK |
| | | | SVTSGLGYMHCCLNPLLYAF |
| | | | VGVKFRERMWMLLLRLGCPN |
| | | | QRGLQRQPSSSRRDSSWSET |
| | | | SEASYSGL |
| Peptide 1 | | 8 | MVLEVSDHQVLNDAEVAALL |
| | | | ENF |
| Peptide 2 | | 9 | NDAEVAALLENFSSSYDYGE |
| | | | NE |
| Peptide 3 | | 10 | FSSSYDYGENESDSCCTSPP |
| | | | CP |
| Peptide 4 | | 11 | ENESDSCCTSPPCPQDFSLN |

### EXAMPLES

### Example 1

This Example describes a method for producing CXCR3 antibodies.

Monoclonal antibodies reactive with human CXCR3 (hCXCR3) are generated by immunising C57BL/6 mice with 2×10⁷ L1.2/hCXCR3 transfected cells stimulated 20 hours prior to harvest with 5 mM butyric acid and emulsified in Complete Freund's Adjuvant (1st immunization intraperitoneal) or Incomplete Freund's Adjuvant (2nd-6th immunizations intraperitoneal), for a total five to six times at 2-wk intervals. The final immunisation are injected intravenously in PBS. Four days later, the spleen is removed and cells fused with the SP2/0 cell line using standard methods. Hybridomas are grown in DMEM (Gibco/Invitrogen) containing 10% Fetalclone (HyClone), 1×HAT supplement (Sigma Aldrich) plus mouse IL-6. After 10-14 days growth culture supernatant is taken for initial screening.

Monoclonal antibodies reactive with CXCR3 are identified using human CXCR3 transfected L1.2 cells, and untransfected L1.2 cells, or L1.2 cells transfected with unrelated or closely receptors such as CXCR1, CXCR2 or C5aR using immunofluorescent staining and analysis using a FACSCalibur (BD Biosciences). Monoclonal antibody staining of cells is performed using standard procedures as described previously (Lee et al., 2006, Nat. Biotech. 24:1279-1284).

Production of antibodies involved growing hybridomas in tissue culture flasks and harvesting the culture medium. For some experiments, the concentration of antibody in the culture supernatant may be sufficient to proceed without further purification. Production of selected antibodies is scaled up and monoclonal antibodies are purified by protein G chromatography, concentrated and buffer exchanged into PBS. Monoclonal antibody concentration is determined using a total IgG ELISA.

L1.2 transfectants expressing high levels of hCXCR3 are used to immunize mice, and monoclonal antibodies are initially identified via flow cytometry that reacted with L1.2 cells transfected with hCXCR3. To ensure clonality, selected hybridomas were subcloned using dilution plating into a 384-well plate. The specificity of cross-reactivity of the subclones is confirmed by flow cytometry with L1.2/hCXCR3 transfectants and untransfected L1.2 cells.

### Example 2

This Example describes a method for determining receptor binding specificity of CXCR3 antibodies.

To assess reactivity of mAbs against transfected cells, indirect immunofluorescence staining and flow cytometry are used. Cells are washed once with PBS and resuspended in 100 μl PBS containing 2% (wt/vol) BSA and 0.1% (wt/vol) sodium azide (staining buffer) and purified antibody. After 30 min at 4° C., cells are washed twice with staining buffer and resuspended in 50 μl PE-conjugated anti-human IgG (Jackson ImmunoResearch Laboratories) diluted 1:500 in staining buffer for the detection of humanized mAbs or 50 μl PE-conjugated anti-mouse IgG (Jackson ImmunoResearch Laboratories) for the detection of mouse mAbs. After incubating for 20 min at 4° C., cells are washed twice with staining buffer and analyzed on LSRII flow cytometer. 7-AAD staining is used to exclude dead cells.

### Example 3

This Example describes a method for epitope mapping CXCR3 antibodies.

Epitope mapping studies are performed to determine the region within CXCR3 that is recognized by the anti-CXCR3 mAb. Initially, biotinylated peptides corresponding to the N-terminal region and the first, second and third extracellular loops of CXCR3 are used in an ELISA.

Overlapping biotinylated peptides spanning the entire N-terminal region of human CXCR3 are then synthesized and used in more defined epitope mapping studies. Briefly, multiwell plates are coated with streptavidin and washed before the biotinylated peptides are added to separate wells and incubated to facilitate binding of the peptides to the plate. Different anti-mCXCR3 antibodies are tested in parallel with newly developed mAb by adding the respective antibodies to the wells of the plate and incubating the plate. An isotype control and buffer only are included as negative controls. Following washing, appropriate conjugated antibodies are added and the plates are incubated. The plates are washed again and binding of the antibodies to the immobilised peptides is visualised.

### Example 4

This Example describes a method for measuring inhibition of T cell migration by anti-CXCR3 antibodies.

Human CD4+ T cells are spun down and washed in migration medium (MM=RPMI 1640, 0.5% BSA) and resuspended at 10⁷ cells/ml. Tissue culture inserts (Becton Dickinson & Co., Mountain View, Calif.) are placed in each of the wells of 24-well tissue-culture plates, forming an upper and lower chamber separated by a polyethylene terepthalate membrane bearing 3-mm-diameter pores. Chemotactic CXCR3 ligand, CXCL9 (MIG), CXCL10 (IP-10) or CXCL11 (1-TAC), (diluted in assay medium) is added to 600 μl of assay medium in the 24-well tissue culture plates. One million cells in 100 μl are pre-incubated for 30 mins with the antibodies. The purified mAb are added to the upper chamber in the wells and the cells are allowed to migrate through to the lower chamber in an 5% CO2, 37° C., incubator for 4 h. The inserts are removed after migration and the cells are counted by the LSRII cytometer (BD Biosciences). Relative cell counts are obtained by acquiring events for a set time period of 30 seconds.

### Example 5

This Example describes use of a depleting anti-CXCR3 antibody for treating NASH.

### MCD Diet to Induce NASH

C57/BL6 mice were fed with control or MCD diet for 5 weeks. The MCD diet is low in choline and methionine and is used to model the induction and progression of NASH. The MCD diet was as described in the prior art (see for example Machado et al., 2015, PLoS One; 10(5): e0127991).

### Antibody Treatment

After 5 weeks, mice were treated with either isotype control antibody or a depleting anti-CXCR3 antibody.

### Histopathology

Liver tissue was processed and stained using standard procedures.

Haemolysin and eosin staining was used to visualise fatty deposits. Picrosirius red staining was used to visualise collagen deposits (and thereby determine the degree of fibrosis).

Steatosis was scored and the severity was graded, based on the percentage of the total area affected, into the following categories: 0 (<5%), 1 (5-33%), 2 (34-66%) and 3 (>66%).

Inflammation is evaluated by counting the number of inflammatory foci per field using a 100× magnification.

### Measurement of NKT Cells and Intrahepatic CD8 T Cells

### Measurement of NKT and Intrahepatic CD8+ T Cells

Livers were minced and hepatic lymphocytes were purified using a Ficoll gradient. Cells were stained with anti-CD45, anti-TCRbeta; anti-NK1.1; anti-CD8 and anti-CXCR3 antibodies. A Fortessa instrument (BD Biosciences) and FlowJo sofware were used for acquisition and data analysis.

### Measurement of Alanine Aminotransferase Levels in Serum

Alanine Aminotransferase (ALT) activity in the serum was measured using a ALT Activity Colorimetric assay according to THE manufactured protocol (Sigma).

Results: Treatment with a CXCR3 Depleting Antibody Protects Against NASH

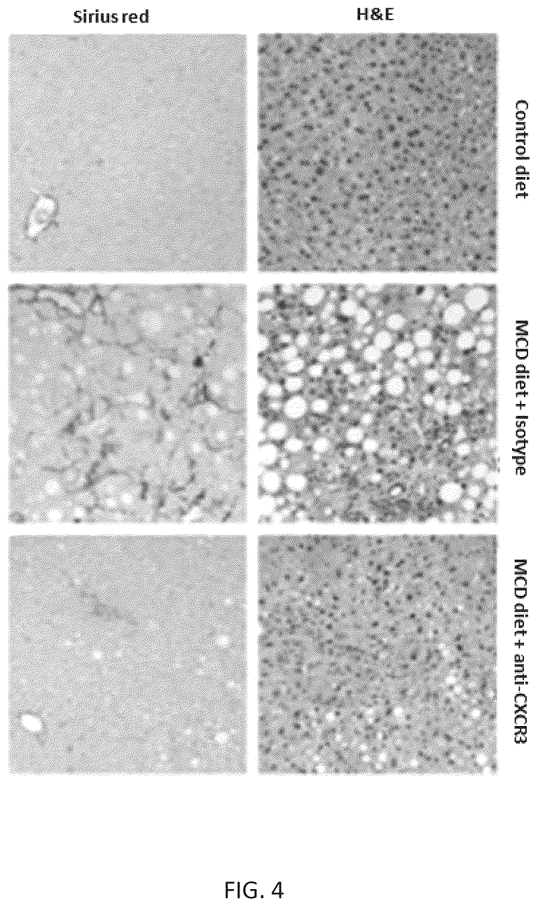

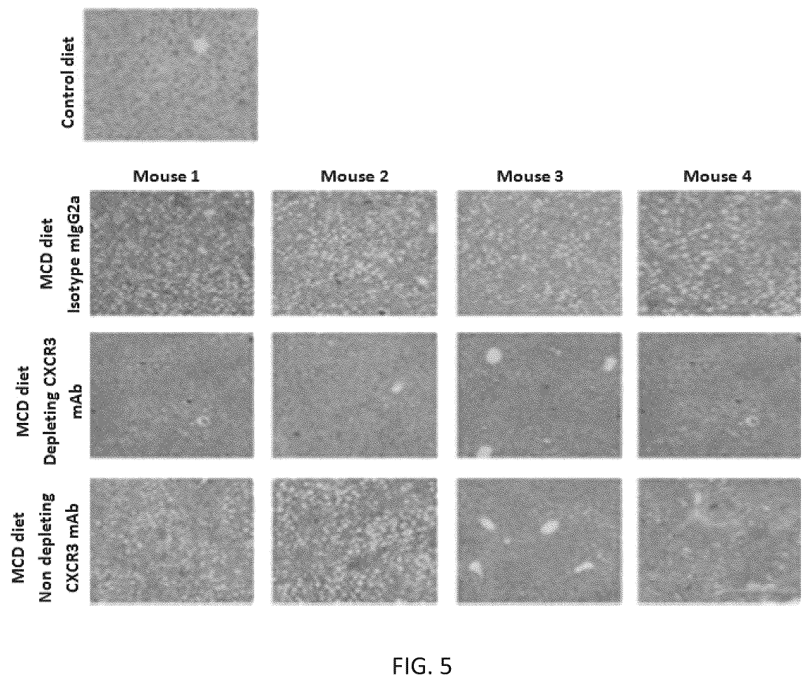

As shown in FIGS. 4 and 5, the livers of mice receiving the MCD diet plus isotype antibody had significant fatty deposits at the conclusion of the trial as compared to mice on the control (normal) diet.

The livers of mice who received an anti-CXCR3 depleting antibody treatment in conjunction with the NASH-inducing MCD diet were significantly protected from the development of fatty deposits.

FIG. 4 also shows that mice receiving an anti-CXCR3 depleting antibody have reduced levels of liver fibrosis compared to mice receiving the isotype antibody in conjunction with the MCD diet.

Thus, the results shown in FIGS. 4 and 5 indicate that treatment with a depleting anti-CXCR3 antibody inhibits liver steatosis and fibrosis in an accepted model of NASH.

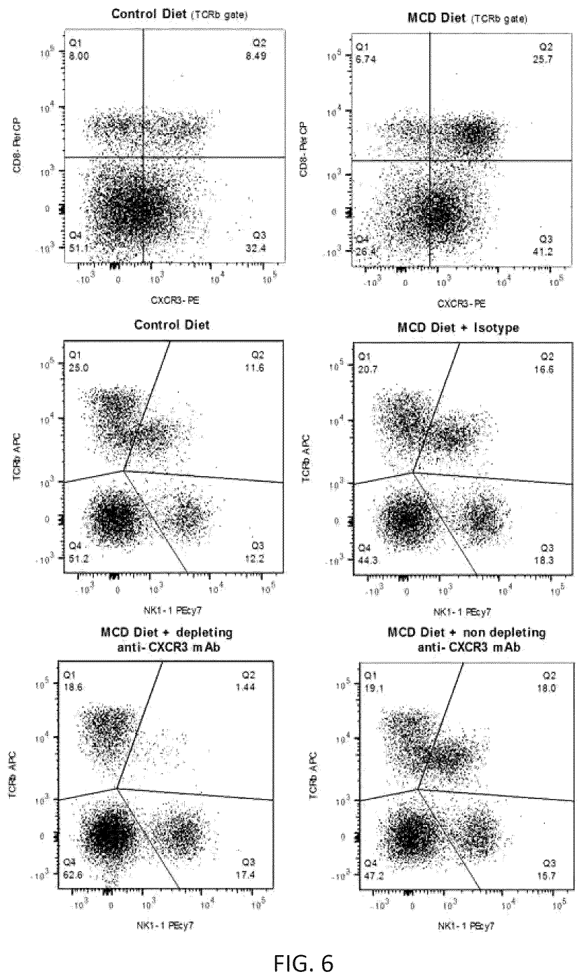

FIG. 6 shows that treatment with an anti-CXCR3 mAbs depletes NKT cells and activated intrahepatic CD8 T-cells. The MCD diet increased the frequency of CD8+ CXCR3+ T-cell and of NKT cells; NKT cells are CXCR3+ and are depleted after treatment with Fc engineered anti-CXCR3 mAB.

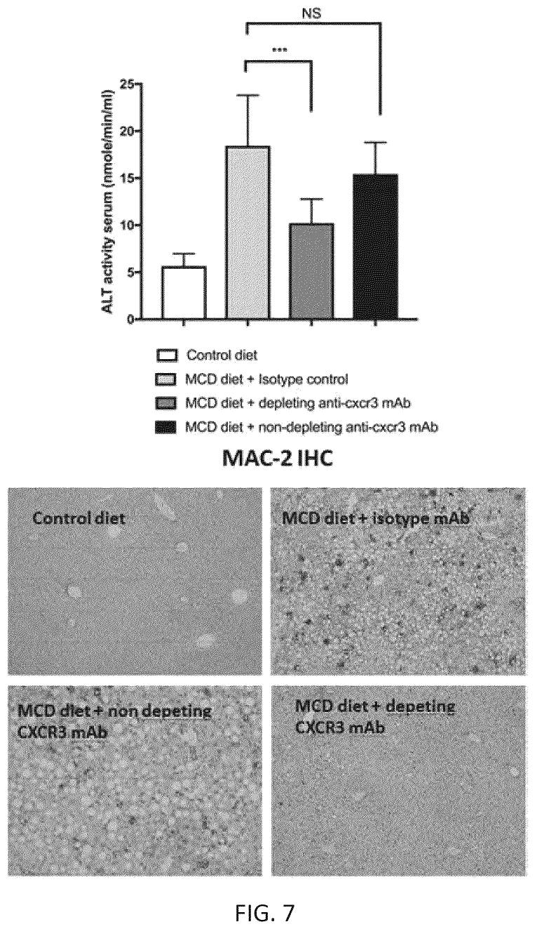

Increased serum levels of ALT are indicative of liver damage. FIG. 7 shows that treatment with anti-CXCR3 antibody decreases the release of alanine aminotransferase into the serum and decreases infiltration of monocytes in liver in an accepted model of NASH.

In summary, these data show that treatment with a CXCR-depleting antibody in the context of a NASH-inducing diet:
inhibits formation of fatty deposits (liver steatosis);
inhibits collagen deposits and fibrosis
depletes NKT cells and activated intrahepatic CD8+ T cells
decreases the release of alanine aminotransferase into the serum indicating an improvement in liver function; and
decreases infiltration of monocytes in the liver.

It will be understood that the invention disclosed and defined in this specification extends to all alternative combinations of two or more of the individual features mentioned or evident from the text or drawings. All of these different combinations constitute various alternative aspects of the invention.

1. (canceled)

2. A method for treating or for delaying the progression of fatty liver disease in a subject, the method comprising, consisting essentially of or consisting of administering to the subject a depleting antigen binding protein that binds to CXCR3, thereby treating or delaying the progression of the fatty liver disease in the subject.

3. (canceled)

4. The method of claim 2, wherein the fatty liver disease is associated, caused by or the result of alcoholism.

5. The method of claim 2, wherein the fatty liver disease is associated, caused by or the result of a non-alcohol dietary cause (non-alcohol fatty liver disease, NAFLD).

6. The method of claim 2, wherein the fatty liver disease is NAFLD and the NAFLD is simple steatosis or simple steatosis including one or more signs of inflammation and fibrosis.

7. The method of claim 2, wherein the fatty liver disease is characterised by liver steatosis, elevated circulating or liver triglycerides and/or elevated circulating or liver cholesterol levels.

8. The method of claim 2, wherein the fatty liver disease is non-alcoholic steatohepatitis (NASH).

9.-13. (canceled)

14. The method of claim 2, wherein the method comprises treating or preventing the accumulating of lipid deposits in the liver of the subject.

15.-16. (canceled)

17. A method of treating or preventing steatohepatitis in a subject, the method comprising administering a depleting antigen binding protein that binds to CXCR3 to the subject, thereby treating or preventing steatohepatitis.

18. The method of claim 17, wherein the steatohepatitis is alcoholic steatohepatitis (ASH) or non-alcoholic steatohepatitis (NASH).

19.-26. (canceled)

27. The method of claim 2, wherein the antigen binding protein binds to or specifically binds to CXCR3 and inhibits CXCR3 activity.

28. The method of claim 2, wherein the antigen binding protein that binds to CXCR3 is a depleting antibody.

29. The method of claim 2, wherein the antigen binding protein is a depleting antibody that has the capacity to cause or mediate a reduction in activity or viability of a cell expressing CXCR3.

30. The method of claim 2, wherein the antigen binding protein is a depleting antibody that has the capacity to induce antibody-dependent cell-mediated cytotoxicity (ADCC).

31. The method of claim 2 wherein the antigen binding protein has been modified to provide the ability to deplete a cell, or has been modified to increase the existing capacity of the antigen binding protein to deplete a cell that expressed CXCR3.

32. The method of claim 2 wherein the antigen binding protein has the ability to deplete a cell selected from: a CXCR3+/CD4+ T cell, CXCR3+/CD8+ T cell and CXCR3+/CD19+ B cell.

33. The method of claim 2, wherein the antigen binding protein inhibits one or more of: ligand binding to CXCR3; ligand induced conformational change of CXCR3; CXCR3 activation; G protein activation; CXCR3 mediated cell signalling; a CXCR3 mediated cell migratory, inflammatory, tumour growth, angiogenic or metastatic response in vitro or in vivo; CXCR3 mediated tumour cell growth; and/or CXCR3 mediated recruitment of inflammatory cells, leukocyte (e.g. neutrophil, eosinophil, mast cell or T cell) migration, integrin activation, chemotactic migration and Th1 cell maturation.

34. (canceled)

35. The method of claim 2, wherein the antigen binding protein does not detectably bind to or bind significantly to CXCR1, CXCR2, and/or C5aR.

36.-37. (canceled)

38. The method of claim 2, wherein the antigen binding protein binds to the first and/or second N-terminal loop in CXCR3.

39. The method of claim 2, wherein the antigen binding protein is in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3;
(iv) one of (i) or (ii) linked to a protein that binds to an immune effector cell;
(v) a diabody;
(vi) a triabody;
(vii) a tetrabody;
(viii) a Fab;
(ix) a F(ab′)₂;
(x) a Fv;
(xi) one of (v) to (ix) linked to a constant region of an antibody, Fc or a heavy chain constant domain (CH) 2 and/or CH3; or
(xii) one of (v) to (ix) linked to a protein that binds to an immune effector cell.

40.-44. (canceled)

45. The method of claim 2, wherein the antigen binding protein comprises an Fc region that is engineered to have enhanced capacity to induce antibody-dependent cell-mediated cytotoxicity (ADCC).

46.-47. (canceled)


SEQUENCE LISTING (11 sequences)

SEQ ID NO: 1 - protein, 330 aa, Homo sapiens [sequence omitted]

SEQ ID NO: 2 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 951 bp, Mus musculus
  source 1..951: mol_type = other DNA
gacagaaccttcctgccagccctctacagcctcctcttcttgctggggctgctaggcaatggggcggtggctgctgtgctactgagtcagcgcactgccctgagcagcacggacaccttcctgctccacctggctgtagccgatgttctgctggtgttaactcttccattgtgggcagtggatgctgctgtccagtgggttttcggccctggcctctgcaaagtggcaggcgccttgttcaacatcaacttctatgcaggggccttcctgctggcttgtataagcttcgacagatatctgagcatagtgcacgccacccagatctaccgcagggacccccgggtacgtgtagccctcacctgcatagttgtatggggtctctgtctgctctttgccctcccagatttcatctacctatcagccaactacgatcagcgcctcaatgccacccattgccagtacaacttcccacaggtgggtcgcactgctctgcgtgtactgcagctagtggctggtttcctgctgccccttctggtcatggcctactgctatgcccatatcctagctgttctgctggtctccagaggccagaggcgttttcgagctatgaggctagtggtagtggtggtggcagcctttgctgtctgctggaccccctatcacctggtggtgctagtggatatcctcatggatgtgggagttttggcccgcaactgtggtcgagaaagccacgtggatgtggccaagtcagtcacctcgggcatggggtacatgcactgctgcctcaatccgctgctctatgcctttgtgggagtgaagttcagagagcaaatgtggatgttgttcacgcgcctgggccgctctgaccagagagggccccagcggcagccgtcatcttcacggagagaatcatcctggtctgagacaactgaggcctcctacctgggcttg

SEQ ID NO: 5 - protein, 367 aa, Mus musculus
MYLEVSERQVLDASDFAFLLENSTSPYDYGENESDFSDSPPCPQDFSLNFDRTFLPALYSLLFLLGLLGNGAVAAVLLSQRTALSSTDTFLLHLAVADVLLVLTLPLWAVDAAVQWVFGPGLCKVAGALFNINFYAGAFLLACISFDRYLSIVHATQIYRRDPRVRVALTCIVVWGLCLLFALPDFIYLSANYDQRLNATHCQYNFPQVGRTALRVLQLVAGFLLPLLVMAYCYAHILAVLLVSRGQRRFRAMRLVVVVVAAFAVCWTPYHLVVLVDILMDVGVLARNCGRESHVDVAKSVTSGMGYMHCCLNPLLYAFVGVKFREQMWMLFTRLGRSDQRGPQRQPSSSRRESSWSETTEASYLGL

SEQ ID NO: 6 - DNA, 1670 bp, Homo sapiens
  source 1..1670: mol_type = other DNA
ccaaccacaagcaccaaagcagaggggcaggcagcacaccacccagcagccagagcaccagcccagccatggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaggccggaatccgggctcccctttcgcccacagtctgacttccccgcattccaggctcctccctccctctgccggctctggctctccccaatatcctcgctcccgggactcactggcagccccagcaccaccaggtctcccgggaagccaccctcccagctctgaggactgcaccattgctgctccttagctgccaagccccatcctgccgcccgaggtggctgcctggagccccactgcccttctcatttggaaactaaaacttcatcttccccaagtgcggggagtacaaggcatggcgtagagggtgctgccccatgaagccacagcccaggcctccagctcagcagtgactgtggccatggtccccaagacctctatatttgctcttttatttttatgtctaaaatcctgcttaaaacttttcaataaacaagatcgtcaggaccaaaaaaaaaaaaaaaaaaaaaaaaaaaaaaaaaaaaaaaaaaaaaaa

SEQ ID NO: 7 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 8 - protein, 23 aa, Homo sapiens
MVLEVSDHQVLNDAEVAALLENF

SEQ ID NO: 9 - protein, 22 aa, Homo sapiens
NDAEVAALLENFSSSYDYGENE

SEQ ID NO: 10 - protein, 22 aa, Homo sapiens
FSSSYDYGENESDSCCTSPPCP

SEQ ID NO: 11 - protein, 20 aa, Homo sapiens
ENESDSCCTSPPCPQDFSLN